(19) United States
(12) Patent Application Publication
(10) Pub. No.: US 2005/0208598 A1
(43) Pub. Date: Sep. 22, 2005

(54) Biotin recognition sensors and high-throughput assays

(76) Inventors: W. Gregory Cox, Eugene, OR (US); Iain D. Johnson, Eugene, OR (US)

Correspondence Address: KOREN ANDERSON;MOLECULAR PROBES, INC., 29851 WILLOW CREEK ROAD, EUGENE, OR, 97402-9132, US

(21) Appl. No.: 11/058,345

(22) Filed: Feb. 14, 2005

Related U.S. Application Data
(60) Provisional application No. 60/544,952, filed on Feb. 13, 2004.

(51) Int. Cl. C12Q 1/68; G01N 33/53; C12M 1/34; C07D 498/02; C07F 5/02

(52) U.S. Cl. 435/7.5; 435/287.2; 548/303.1; 534/727; 544/76; 546/104; 548/110

(57) ABSTRACT

In one embodiment is provided a new class of biotin recognition sensors which comprise a biotin recognition compound (containing a biotin binding moiety), a fluorescent donor moiety, and an acceptor moiety. These compounds are useful for detecting biotin in a sample or on a carrier molecule. In addition to compounds, the invention also provides methods and kits for detecting the presence of biotin in a sample or on a carrier molecule.

### CROSS-REFERENCE TO RELATED APPLICATIONS

This application claims priority of U.S. Ser. No. 60/544,952, filed Feb. 13, 2004, which disclosure is herein incorporated by reference.

### FIELD OF THE INVENTION

The disclosure relates to methods and biotin recognition sensor (BRS) compositions for the detection of biotin molecules on a carrier molecule. The disclosure has applications in the fields of molecular biology, cell biology, immunohistochemistry, diagnostics and therapeutics.

### BACKGROUND OF THE INVENTION

Some of the strongest affinities known in the biological world exist between biotin and certain biotin recognition compounds (“BRC”). For example, the dissociation constant between BRC avidin and biotin is on the order of 10⁻¹⁵ M. A second BRC, streptavidin, has a dissociation constant with biotin of approximately 10⁻¹⁴ M. This affinity is substantially maintained even when biotin is derivatized with carrier molecules such as proteins or nucleic acids. These affinities have been utilized in numerous fields of biology and biotechnology since the pioneering work involving avidin and biotin by Dr. Edward Bayer and Dr. Meier Wilchek in the 1970's. Originally applied to purification and localization procedures for biologically active macromolecules, BRC-biotin technology today has widespread use in medical diagnostics. Newer applications which continue to be developed include affinity targeting, cell cytometry, blotting technology, drug delivery, hybridoma technology, human stem cell selection and reinfusion as well as several approaches to enzyme capture. In some applications, the BRC is immobilized onto an inert material over which a solution containing biotinylated carrier molecules is passed. The affinity of the biotin for the BRC provides for the separation of the carrier molecule from the solution. A review of this technology, with a focus on avidin, can be found in Applications of Avidin-Biotin Technology to Affinity-Based Separation, Bayer, et al., J. of Chromatography, p. 3-11 (1990).

Despite the extensive use of biotinylated carrier molecules in a variety of applications, descriptions of how to ascertain the extent of carrier molecule biotinylation are sparse. Of the few that are known, perhaps the most common involves a titration with 4-hydroxyazobenzene-2-carboxylic acid (HABA) (Bayer, E. A. et al., Protein Biotinylation. Methods Enzymol. 184:138-160 (1990)). In this case, HABA binds to avidin to give an absorption maximum at 500 nm. When biotin or biotinylated proteins are added, biotin displaces HABA from avidin and the absorbance at 500 nm is reduced. The decrease in absorbance is then used to determine the extent of biotinylation. Because this method is based on an absorbance measurement, the HABA titration suffers from low sensitivity. As a result, amounts typically in the order of several nanomoles of protein are required for the determination of the degree of biotinylation. More sensitive methods for biotin determination should reduce the amount of biotinylated protein sacrificed for this purpose.

Another measure of the extent of biotinylation on a protein is provided by Rao et al., Bioconjugate Chem. 8: 94-98 (1997). In this paper, fluorescein isothiocyanate is attached to avidin, which alters its fluorescent profile upon binding biotin. While the use of fluorescein increases the sensitivity of this assay, an HPLC separation step is required which prevents the rapid detection of a group of samples.

Consequently, there remains a need for a fluorescent, high-throughput method for determining the extent of biotinylation on a carrier molecule or in a sample. The present invention addresses this and other needs.

### SUMMARY OF THE INVENTION

In a first aspect is provided a biotin recognition sensor (BRS) comprising a biotin recognition compound (BRC) comprising a biotin-binding moiety. The invention also comprises a donor moiety, wherein said donor moiety is covalently attached to the BRC at any portion other than within the biotin-binding moiety. The invention also comprises a biotin mimic (acceptor moiety), wherein the biotin mimic (acceptor moiety) is noncovalently attached to the BRC at the biotin-binding moiety.

In an exemplary embodiment, the BRC is avidin, streptavidin, captavidin, neutravidin, anti-biotin antibody or fragment thereof. In another embodiment the biotin mimic is (2-(4′-hydroxyazobenzene)benzoic acid) (HABA), Strep-Tag peptides or desthiobiotin. In another exemplary embodiment the acceptor moiety comprises substituted (2-(4′-hydroxyazobenzene)benzoic acid) (HABA), unsubstituted (2-(4′-hydroxyazobenzene)benzoic acid) (HABA), a quencher moiety or a dye moiety. Thus, in one embodiment, the acceptor moiety is covalently bonded to the biotin mimic moiety. In one aspect, the biotin mimic comprises a dye labeled desthiobiotin or a quencher labeled desthiobiotin. In a further aspect, the biotin mimic is desthiobiotin labeled with and acceptor moiety such as Alexa Fluor 555. In another embodiment the acceptor moiety is HABA.

In an exemplary embodiment the donor moiety is a xanthene, cyanine, borapolyazaindacene (BODIPY), coumarin, oxazine, acridinone, or styryl dyes. In one aspect the donor moiety is Alexa Fluor 488 dye or fluorescein. In a further aspect, the donor dye is encapsulated in a microparticle. In a particular useful embodiment the BRS comprises avidin as the BRC, HABA as the biotin mimic and acceptor moiety and Alexa Fluor 488 dye as the donor moiety. In another equally useful embodiment the BRS comprises Streptavidin as the BRC, desthiobiotin as the biotin mimic, which is covalently conjugated to the acceptor moiety of Alexa Fluor 555 dye and the donor moiety is Alexa Fluor 488 dye.

In a second aspect is provided a method of detecting biotin on a carrier molecule. The method comprises:
i) contacting the carrier molecule with a BRS, to form a carrier molecule-biotin-BRS complex, wherein the BRS comprises:
   a) a biotin recognition compound (BRC) comprising a biotin-binding moiety;
   b) a donor moiety, wherein the donor moiety is covalently attached to the BRC at any portion other than within the biotin-binding moiety; and
   c) a biotin mimic compound that comprises an acceptor moiety, wherein the biotin mimic compound is non-covalently attached to the BRC at the biotin-binding moiety;
ii) illuminating the carrier molecule-biotin-BRS complex with an appropriate wavelength to form an illuminated carrier molecule-biotin-BRS complex; and,
iii) observing the illuminated carrier molecule-biotin-BRS complex, whereby the biotin on a carrier molecule is detected.

In an exemplary embodiment, the BRC is avidin, streptavidin, captavidin, neutravidin, anti-biotin antibody or fragment thereof. In another embodiment the biotin mimic is (2-(4′-hydroxyazobenzene)benzoic acid) (HABA), Strep-Tag peptides or desthiobiotin. In another exemplary embodiment the acceptor moiety comprises substituted (2-(4′-hydroxyazobenzene)benzoic acid) (HABA), unsubstituted (2-(4′-hydroxyazobenzene)benzoic acid) (HABA), a quencher moiety or a dye moiety. Thus, in one embodiment, the acceptor moiety is covalently bonded to the biotin mimic moiety. In one aspect, the biotin mimic comprises a dye labeled desthiobiotin or a quencher labeled desthiobiotin. In a further aspect, the biotin mimic is desthiobiotin labeled with and acceptor moiety such as Alexa Fluor 555. In another embodiment the acceptor moiety is HABA.

In an exemplary embodiment the donor moiety is a xanthene, cyanine, borapolyazaindacene (BODIPY), coumarin, oxazine, acridinone, or styryl dyes. In one aspect the donor moiety is Alexa Fluor 488 dye or fluorescein. In a further aspect, the donor dye is encapsulated in a microparticle. In a particular useful embodiment the BRS comprises avidin as the BRC, HABA as the biotin mimic and acceptor moiety and Alexa Fluor 488 dye as the donor moiety. In another equally useful embodiment the BRS comprises Streptavidin as the BRC, desthiobiotin as the biotin mimic, which is covalently conjugated to the acceptor moiety of Alexa Fluor 555 dye and the donor moiety is Alexa Fluor 488 dye.

In a third aspect is provided a method of determining the number of biotin molecules on a carrier molecule. This method comprises:
i) releasing biotin from the carrier molecule;
ii) contacting the biotin with a BRS, whereby said biotin displaces an acceptor moiety on the BRS, thereby forming a biotin-BRS complex, wherein the BRS comprises:
   a) a biotin recognition compound (BRC) comprising a biotin-binding moiety;
   b) a donor moiety, wherein the donor moiety is covalently attached to the BRC at any portion other than within the biotin-binding moiety; and
   c) a biotin mimic compound that comprises an acceptor moiety, wherein the biotin mimic compound is non-covalently attached to the BRC at the biotin-binding moiety;
iii) illuminating the biotin-BRS complex with an appropriate wavelength to form an illuminated sample; and,
iv) observing the illuminated sample whereby the number of biotin molecules is determined.

The method also comprises contacting the biotin with a BRS, whereby said biotin displaces an acceptor moiety on said BRS, thereby forming a biotin-BRS complex. The method also comprises detecting the biotin-BRS complex, whereby said number of biotin molecules is determined. In another exemplary embodiment, the detecting comprises quantifying the number of biotins present on a carrier molecule. In yet another exemplary embodiment, the carrier molecule is a member selected from a peptide and a protein. In still another exemplary embodiment, the releasing further comprises contacting said carrier molecule with a protease. In another exemplary embodiment, the carrier molecule is a member selected from DNA and RNA. In another exemplary embodiment, the releasing further comprises contacting said carrier molecule with a nuclease. In an exemplary embodiment, the BRC is avidin, streptavidin, captavidin, neutravidin, anti-biotin antibody or fragment thereof. In another embodiment the biotin mimic is (2-(4′-hydroxyazobenzene)benzoic acid) (HABA), Strep-Tag peptides or desthiobiotin. In another exemplary embodiment the acceptor moiety comprises substituted (2-(4′-hydroxyazobenzene)benzoic acid) (HABA), unsubstituted (2-(4′-hydroxyazobenzene)benzoic acid) (HABA), a quencher moiety or a dye moiety. Thus, in one embodiment, the acceptor moiety is covalently bonded to the biotin mimic moiety. In one aspect, the biotin mimic comprises a dye labeled desthiobiotin or a quencher labeled desthiobiotin. In a further aspect, the biotin mimic is desthiobiotin labeled with and acceptor moiety such as Alexa Fluor 555. In another embodiment the acceptor moiety is HABA.

In an exemplary embodiment the donor moiety is a xanthene, cyanine, borapolyazaindacene (BODIPY), coumarin, oxazine, acridinone, or styryl dyes. In one aspect the donor moiety is Alexa Fluor 488 dye or fluorescein. In a further aspect, the donor dye is encapsulated in a microparticle. In a particular useful embodiment the BRS comprises avidin as the BRC, HABA as the biotin mimic and acceptor moiety and Alexa Fluor 488 dye as the donor moiety. In another equally useful embodiment the BRS comprises Streptavidin as the BRC, desthiobiotin as the biotin mimic, which is covalently conjugated to the acceptor moiety of Alexa Fluor 555 dye and the donor moiety is Alexa Fluor 488 dye.

In a fourth aspect, the invention provides a kit for detecting biotin on a carrier molecule or in a sample, wherein said kit comprises a BRS, and instructions on the use of said BRS. In an exemplary embodiment, the kit further comprises a reaction buffer. In another exemplary embodiment, the kit further comprises biotin as a positive control. In another exemplary embodiment, the carrier molecule is a member selected from peptide and a protein. In another exemplary embodiment, the kit is further comprises a protease. In another exemplary embodiment, the carrier molecule is a member selected from DNA and RNA. In another exemplary embodiment, kit further comprises a nuclease.

In a fifth aspect, the invention provides a composition for the quantitative determination of biotin in a sample, wherein the composition comprises a sample and a BRS, and wherein said BRS reacts with said biotin to form an amount of a fluorescent product proportional to the amount of biotin in said sample.

### BRIEF DESCRIPTION OF THE DRAWINGS

FIG. 1 is a biotin standard curve.

FIG. 2 is a series of biotin standard curves that have varied analytical ranges due to the presence of unlabeled avidin in the sample. The first curve (square) contained no added unlabeled avidin. The second curve (circle) had 0.8 μM unlabeled avidin. The third curve (diamond) had 7.0 μM unlabeled avidin.

FIG. 3 is a comparison of the sensitivity of the biotin assay when the sample or protein is treated to enzymatic digestion. The dotted line indicates the intersection of predicted and measured values. The square line represents a sample treated with proteinase K while the circle line represents a sample that was not treated with proteinase K.

FIG. 4 is independent determination of the extent of biotinylation through the use of MALDI-TOF mass spectrometry. The line designated as “145.2” is not labeled with biotin. The line designated as “148.8” is labeled with biotin.

FIG. 5 is a comparison of the sensitivity of the biotin assay when the sample or nucleic acid is treated to enzymatic digestion. The dotted line indicates the intersection of predicted and measured values. The square line represents a sample treated with micrococcal nuclease while the circle line represents a sample that was not treated with micrococcal nuclease.

FIG. 6 is a biotin standard curve obtained using a detection complex comprising 200 nM Alexa Fluor 488-labeled avidin and 1 μM Alexa Fluor 555-labeled desthiobiotin.

### DETAILED DESCRIPTION OF THE INVENTION

Introduction

There is a continuous and expanding need for rapid, highly specific methods of detecting and quantifying chemical, biochemical and biological analytes in research and diagnostic mixtures. Of particular value are methods for measuring small quantities of small organic molecules, nucleic acids, peptides (e.g., enzymes), pharmaceuticals, metabolites, and other materials of diagnostic value.

The invention provides a new class of biotin recognition sensors (“BRSs”). These complexes comprise a biotin recognition compound (BRC), which further comprises one or more biotin binding moieties. The BRS also comprises a donor moiety and a biotin mimic which is or comprises an acceptor moiety. In an exemplary embodiment, the donor moiety is fluorescent and covalently attached to the BRC at any portion outside of the biotin-binding moiety. In another exemplary embodiment, the biotin mimic compound comprises an acceptor moiety is noncovalently attached to the biotin-binding moiety of the BRC. In another embodiment, the acceptor moiety is covalently bonded to the BRC. When a BRS as described above is contacted with biotin, either attached to a carrier molecule or unattached in solution, the biotin dislodges the biotin mimic and/or acceptor moiety, thus producing a detectable response, such as fluorescence, from the donor moiety in the newly formed carrier molecule-biotin-BRS complex or biotin-BRS complex. Use of a fluorogenic donor moiety in the BRS has several important advantages. First, BRSs containing a fluorogenic donor moiety are relatively inexpensive to synthesize and purify. Second, due to the intensity of the fluorogenic donor moiety, only small amounts of the biological sample or carrier molecule are required to perform the biotin assay for the target enzyme. Finally, due to the homogenous nature of the assay, little or no purification of the biological sample or carrier molecule is required, no washing steps are required and measurements can be rapidly accomplished.

Definitions

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a compound” includes a plurality of compounds and reference to “an conjugate” includes a plurality of conjugates and the like.

Unless defined otherwise, all technical and scientific terms used herein have the same meaning as commonly understood by one of ordinary skill in the art to which this invention is related. The following terms are defined for purposes of the invention as described herein. Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), Vogel's Encyclopedia of Practical Organic Chemistry, 5th ed., Longman Scientific and Technical Ltd., Essex, 1991, pp. 809-816; and Heller, Acc. Chem. Res. 23:128 (1990).

The compounds of the present invention may also contain unnatural proportions of atomic isotopes at one or more of the atoms that constitute such compounds. For example, the compounds may be radiolabeled with radioactive isotopes, such as, for example, tritium (³H), iodine-125 (¹²⁵I) or carbon-14 (¹⁴C). All isotopic variations of the compounds of the present invention, whether radioactive or not, are intended to be encompassed within the scope of the present invention.

The term “aqueous solution” as used herein refers to a solution that is predominantly water and retains the solution characteristics of water. Where the aqueous solution contains solvents in addition to water, water is typically the predominant solvent.

The term “detectable response” as used herein refers to an occurrence of or a change in, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters.

The term “carrier molecule” as used herein refers to a biological or a non-biological component that is covalently bonded to biotin. Such components include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. For the purposes of this invention, the biotin-recognition molecule is not considered a carrier molecule.

The term “linker” or “L”, as used herein, refers to a single covalent bond or a series of stable covalent bonds incorporating 1-20 nonhydrogen atoms selected from the group consisting of C, N, O, S and P that covalently attach the fluorogenic or fluorescent compounds to another moiety such as a chemically reactive group or a biological and non-biological component. Exemplary linking members include a moiety that includes —C(O)NH—, —C(O)O—, —NH—, —S—, —O—, and the like. The linker can be used to attach the compound to another component of a conjugate, such as a carrier molecule.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with biotin, a fluorophore or another moiety.

The term, “peptide”, as used herein, refers to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “lipid” refers to a group of organic compounds that are esters or ethers such as fatty aliphatic acid esters, and are characterized by being insoluble in water but soluble in many organic solvents. They are usually divided in at least three classes: (1) “simple lipids” which include fats and oils as well as waxes; (2) “compound lipids” which include phospholipids and glycolipids; (3) “derived lipids” such as steroids.

The term “sample,” as used herein, refers to any material that may contain a compound possessing a biotin moiety. The sample may also include diluents, buffers, detergents, and contaminating species, debris and the like that are found mixed with the target. Illustrative examples include urine, sera, blood plasma, total blood, saliva, tear fluid, cerebrospinal fluid, secretory fluids from nipples and the like. Also included are solid, gel or sol substances such as mucus, body tissues, cells and the like suspended or dissolved in liquid materials such as buffers, extractants, solvents and the like. Typically, the sample is a live cell, a biological fluid that comprises endogenous host cell proteins, nucleic acid polymers, nucleotides, oligonucleotides, peptides and buffer solutions. The sample may be in an aqueous solution, a viable cell culture or immobilized on a solid or semi solid surface such as a polyacrylamide gel, membrane blot or on a microarray.

The term, “biotin-recognition sensor” (“BRS”), as used herein, refers to a complex of molecules that are capable of binding biotin and generating a detectable response. A BRS comprises a biotin recognition compound (BRC), a donor moiety, and an acceptor moiety, wherein either the acceptor or the donor is or comprises a biotin mimic.

The term, “biotin-recognition compound” (“BRC”), as used herein, refers to a molecule that contains one or more biotin-binding moieties. Examples of BRCs include streptavidin and streptavidin derivatives, avidin and avidin derivatives, captavidin and captavidin derivatives, and neutravidin and neutravidin derivatives. Other examples include antibodies raised against biotin, such as Mouse IgG antibody. BRCs encompass monoclonal antibodies, as well as biotin-binding antibodies which are constituents of polyclonal sera.

The term, “biotin-binding moiety”, as used herein, refers to the portion of the biotin-recognition compound which primarily interacts with the biotin or biotin derivative. Prior to interacting with the biotin, this moiety noncovalently interacts with the acceptor moiety.

The term, “biotin-BRS complex”, as used herein, refers to a BRS in which one or more of its acceptor moieties has been replaced with one or more biotin molecules.

The term, “carrier molecule-biotin-BRS complex”, as used herein, refers to a BRS in which one or more of its acceptor moieties has been replaced with one or more biotin molecules which are conjugated to a carrier molecule.

The term, “biotin mimic”, as used herein, refers to a molecule that binds to a biotin-binding moiety and remains bound under assay conditions. This binding may be covalent or non-covalent.

The term, “energy transfer”, as used herein, refers to the process by which the excited state energy of an excited group, such as a donor, is altered by a modifying group, such as an acceptor. If the excited state energy-modifying group is a quenching group, then the fluorescence emission from the fluorescent group is attenuated (quenched). Energy transfer can occur through fluorescence resonance energy transfer, or through direct energy transfer. The exact energy transfer mechanisms in these two cases are different. It is to be understood that any reference to energy transfer in the instant application encompasses all of these mechanistically-distinct phenomena.

As used herein, “energy transfer pair” refers to any two molecules that participate in energy transfer. Typically, one of the molecules acts as a fluorescent group, and the other acts as a fluorescence-modifying group. The preferred energy transfer pair of the instant invention comprises a fluorescent group and a quenching group of the invention. There is no limitation on the identity of the individual members of the energy transfer pair in this application. All that is required is that the spectroscopic properties of the energy transfer pair as a whole change in some measurable way if the distance between the individual members is altered by some critical amount. “Energy transfer pair” is used to refer to a group of molecules that form a complex within which energy transfer occurs. Such complexes may include, for example, two fluorescent groups, which may be different from one another and one quenching group, two quenching groups and one fluorescent group, or multiple fluorescent groups and multiple quenching groups. In cases where there are multiple fluorescent groups and/or multiple quenching groups, the individual groups may be different from one another.

The term “donor” as used herein refers to a fluorescent compound that emits light to produce an observable detectable signal.

As used herein, “acceptor” or “quenching group” refers to any moiety that can attenuate fluorescence in a proximity dependent manner. Examples of acceptor moieties include fluorescent dyes, non-fluorescent dyes, gold particles, nitrophenyl compounds, nitroxide spin label-modifying groups.

“FET,” as used herein, refers to “Fluorescence Energy Transfer.” “FRET,” as used herein, refers to “Fluorescence Resonance Energy Transfer.” These terms are used herein to refer to both radioactive and non-radioactive energy transfer processes. For example, processes in which a photon is emitted and those involving long range electron transfer are included within these terms. Throughout this specification, both of these phenomena are subsumed under the general term “donor-acceptor energy transfer.”

Compounds

The present invention provides biotin recognition sensors (BRS) useful for the detection of biotin. The compounds include a biotin recognition compound (BRC) comprising a biotin-binding moiety; a donor moiety, wherein said donor moiety is covalently attached to said BRC at any portion other than within said biotin-binding moiety; and a biotin mimic comprising an acceptor moiety, where the acceptor moiety or biotin mimic is noncovalently attached to the BRC through its biotin-binding moiety. In an alternative embodiment, the BRS comprises a) a biotin recognition compound (BRC) comprising a biotin-binding moiety; b) an acceptor moiety, wherein the acceptor moiety is covalently attached to the BRC at any portion other than within the biotin-binding moiety; and c) a biotin mimic compound that comprises a donor moiety, wherein the biotin mimic compound is non-covalently attached to the BRC at the biotin-binding moiety. The components of the compounds of the invention are disclosed in greater detail below.

a) Biotin Recognition Compounds (BRC)

A variety of biotin recognition compounds can be utilized in this invention. These compounds can have a variety of sizes and molecular weights. In an exemplary embodiment, a BRC can have a molecular weight of between 500 Da to 50 kDa. In an exemplary embodiment, the BRC is avidin. In another exemplary embodiment, the BRC is streptavidin. In another exemplary embodiment, the BRC is an anti-biotin antibody or fragment thereof. In another exemplary embodiment, the BRC is an egg yolk biotin-binding protein (BBP-1 and BBP-2) as described in White HB, Biochem. J. 241:677-684 (1987). Further explanation of these BRCs, as well as the description of other examples, are described in section 7.6 of Haugland, Molecular Probes, Inc. Handbook of Fluorescent Probes and Research Chemicals, (9^{th} ed., including the CD-ROM, September 2002).

The BRC can contain any number of biotin-binding moieties greater than one that still maintains the structural and functional integrity of the BRC. In an exemplary embodiment, the BRC is avidin or streptavidin, which typically comprise four biotin-binding moieties. In one aspect these BRC are conjugated to a microparticle such as a microspheres, which encapsulate either the donor or acceptor dye. The microparticle coated avidin or steptavidin would likely decrease the number of biotin-binding moieties.

The BRC can also be modified from its natural state. In some instances, the biotin-binding moiety can be chemically altered. In an exemplary embodiment, a tyrosine moiety in the avidin biotin binding moiety is nitrated. In other instances, some of the biotin-binding moieties are either inactivated or made inaccessible for interactions with biotin. In another exemplary embodiment, an avidin BRC is conjugated to an acrylamide polymer that blocks access to one or more biotin-binding moieties. For further examples, see Bayer et al., U.S. Pat. No. 5,973,124.

b) Biotin-Mimic Moiety

In the BRS, the moiety which interacts with the biotin-binding moiety (biotin mimic) must possess certain properties. First, the moiety must be able to bind in the biotin binding moiety of the BRC. In addition, the binding affinity of the moiety to the biotin-binding moiety should be such that the moiety is readily displaced by biotin. In an exemplary embodiment, the moiety is an acceptor moiety, and must therefore noncovalently interact with the biotin-binding moiety in such a way that it is readily displaced by biotin.

The biotin mimic must be able to bind in the biotin binding moiety of the BRC. The acceptor moiety's absorption pattern may be either intrinsic (e.g. HABA) or obtained by stable covalent attachment of a dye label (e.g. Alexa Fluor 555 dye-labeled desthiobiotin). The ligand may be either fluorescent (e.g. Alexa Fluor 555 dye-labeled desthiobiotin) or nonfluorescent. (e.g. HABA). Examples of biotin moieties of use in this invention include HABA, desthiobiotin (Hirsch et al., Anal. Biochem. 308:343-357 (2002)), and Strep-tag-II (Schmidt et al., J Mol Biol. 255:753-766 (1996)).

For the purposes of the present disclosure the biotin mimic functions as either the acceptor or donor moiety or is covalently bonded to the acceptor or donor moiety. Thus in one embodiment, HABA is the biotin mimic and also functions as the acceptor moiety. In another embodiment, desthiobiotin is the biotin-mimic and is conjugated to an acceptor such as Alexa Fluor 555 dye, which acts as a quencher.

c) Donor Moiety and Acceptor Moiety

The BRS further comprises donor and acceptor moieties. These moieties are attached to the BRC in different ways, depending on the type of assay utilized. In an exemplary embodiment, the donor moiety is covalently attached to the BRC at any portion other than within said biotin-binding moiety, and the acceptor moiety is noncovalently attached to the BRC at said biotin-binding moiety. In another exemplary embodiment, the donor moiety is noncovalently attached to the BRC at said biotin-binding moiety, and said acceptor moiety is covalently attached to the BRC at any portion other than within said biotin-binding moiety.

The donor and acceptor moieties of use in the invention are subject to two constraints. First, they must possess favorable spectroscopic or optical properties. Second, they must possess favorable properties with respect to the biotin-binding moiety.

Thus, donor moiety and acceptor moiety is independently a fluorophore, a quenching moiety or a fluorescent protein. A fluorophore of the present invention is any chemical moiety that exhibits an absorption maximum beyond 280 nm. Fluorophores of the present invention include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Ser. Nos. 09/968,401 and 09/969,853), a carbocyanine (including any corresponding compounds in U.S. Ser. Nos. 09/557,275; 09/969,853 and 09/968,401; U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

When the fluorophore is a xanthene, the fluorophore is optionally a fluorescein, a rhodol (including any corresponding compounds disclosed in U.S. Pat. Nos. 5,227,487 and 5,442,045), or a rhodamine (including any corresponding compounds in U.S. Pat. Nos. 5,798,276; 5,846,737; U.S. Ser. No. 09/129,015). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Preferred fluorophores of the invention include xanthene (rhodol, rhodamine, fluorescein and derivatives thereof) coumarin, cyanine, pyrene, oxazine and borapolyazaindacene. Most preferred are fluorinated xanthenes, fluorinated coumarins and cyanines, including dyes sold under the tradename ALEXA FLUOR and OREGON GREEN.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

In one aspect of the invention, the fluorophore has an absorption maximum beyond 480 nm. In a particularly useful embodiment, the fluorophore absorbs at or near 488 nm to 514 nm (particularly suitable for excitation by the output of the argon-ion laser excitation source) or near 546 nm (particularly suitable for excitation by a mercury arc lamp).

The above disclosed fluorophores may also function as quenching moieties depending on the environment of the chemical moiety, wherein the moiety absorbs light but does not re-emit light at a different wavelength. When coupled with a donor moiety some fluorophores function as quenching moieties.

Fluorescent or luminescent proteins also find use as donor or acceptor moieties for the BSR compounds of the present invention. Examples of fluorescent proteins include green-fluorescent protein (GFP), aquorin and the phycobiliproteins and the derivatives thereof. Particularly useful fluorescent proteins are the phycobiliproteins disclosed in U.S. Pat. Nos. 4,520,110; 4,859,582; 5,055,556 and the fluorophore bilin protein combinations disclosed in U.S. Pat. 4,542,104.

The donor moiety for the BRS may be any dye that can be attached to the biotin-binding moiety by a stable covalent linkage such as (but not limited to) a carboxamide or thioether linkage. The number of donor moieties per BRC can be any number that maintains the structural and functional integrity of the BRC. In an exemplary embodiment, the number of donor moieties is three. The donor also should not exhibit any change in detectable response upon addition of biotin in the absence of the acceptor moiety. In an exemplary embodiment, the donor moiety is a xanthene including Alexa Fluor 488.

i) Favorable Spectroscopic or Optical Properties

There is a great deal of practical guidance available in the literature for selecting appropriate donor-acceptor pairs for particular probes, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties for choosing reporter-acceptor pairs (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing reporter and acceptor molecules for covalent attachment via common reactive groups that can be added to a nucleic acid, as exemplified by the following references: Haugland (supra); Ullman et al., U.S. Pat. No. 3,996,345; Khanna et al., U.S. Pat. No. 4,351,760. Thus, it is well within the abilities of those of skill in the art to choose an energy exchange pair for a particular application and to conjugate the members of this pair to a probe molecule, such as, for example, a nucleic acid, peptide or other polymer. Known energy transfer pairs include those disclosed in U.S. Pat. Nos. 6,358,684; 5,863,727; 6,372,445 and 5,656,554.

Generally, it is preferred that an absorbance band of the acceptor substantially overlap the fluorescence emission band of the donor. When the donor (fluorophore) is a component of a probe that utilizes donor-acceptor energy transfer, the donor fluorescent moiety and the quencher (acceptor) of the invention are preferably selected so that the donor and acceptor moieties exhibit donor-acceptor energy transfer when the donor moiety is excited. One factor to be considered in choosing the fluorophore-acceptor pair is the efficiency of donor-acceptor energy transfer between them. Preferably, the efficiency of FRET between the donor and acceptor moieties is at least 10%, more preferably at least 50% and even more preferably at least 80%. The efficiency of FRET can easily be empirically tested using the methods both described herein and known in the art.

The efficiency of energy transfer between the donor-acceptor pair can also be adjusted by changing the ability of the donor and acceptor groups to dimerize or closely associate. If the donor and acceptor moieties are known or determined to closely associate, an increase or decrease in association can be promoted by adjusting the length of a linker moiety, or of the probe itself, between the donor and acceptor. The ability of donor-acceptor pair to associate can be increased or decreased by tuning the hydrophobic or ionic interactions, or the steric repulsions in the probe construct. Thus, intramolecular interactions responsible for the association of the donor-acceptor pair can be enhanced or attenuated. Thus, for example, the association between the donor-acceptor pair can be increased by, for example, utilizing a donor bearing an overall negative charge and an acceptor with an overall positive charge.

Reactive Groups

In another embodiment of the invention, the compounds of the invention are chemically reactive, and are substituted by at least one reactive group. This reactive group can be represented by either Rₓ, which represents a reactive functional moiety, or (-L-Rₓ), which represents a reactive functional moiety Rₓ that is attached to the BRS compound by a covalent linkage L. The reactive group functions as the site of attachment for another moiety, such as a solid support, wherein the reactive group chemically reacts with an appropriate reactive or functional group on the solid support. Thus, in another aspect of the present invention the BRS comprises a BRC, donor moiety, biotin mimic (acceptor moiety), and a reactive group moiety.

In an exemplary embodiment, the compounds of the invention further comprise a reactive group which is a member selected from an acrylamide, an activated ester of a carboxylic acid, an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine, a hydrazide, an imido ester, an isocyanate, an isothiocyanate, a maleimide, a phosphoramidite, a reactive platinum complex, a sulfonyl halide, a thiol group, and a photoactivatable group.

These reactive groups are synthesized during the formation of the BRS to provide BRS-reactive group-containing compounds. In this way, BRS compounds incorporating a reactive group can be covalently attached to a wide variety of solid supports that contain or are modified to contain functional groups with suitable reactivity, resulting in chemical attachment of the components. In an exemplary embodiment, the reactive group of the BRSs of the invention and the functional group of the solid support comprise electrophiles and nucleophiles that can generate a covalent linkage between them. Alternatively, the reactive group comprises a photoactivatable group, which becomes chemically reactive only after illumination with light of an appropriate wavelength. Typically, the conjugation reaction between the reactive group and the solid support results in one or more atoms of the reactive group being incorporated into a new linkage attaching the BRS compound of the invention to the solid support. Selected examples of functional groups and linkages are shown in Table 1, where the reaction of an electrophilic group and a nucleophilic group yields a covalent linkage.

TABLE 1
Examples of some routes to useful covalent linkages
with electrophile and nucleophile reactive groups

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |
| *Activated esters, as understood in the art, generally have the formula —COΩ, where Ω is a good leaving group (e.g. oxysuccinimidyl (—OC₄H₄O₂) oxysulfosuccinimidyl (—OC₄H₃O₂—SO₃H), -1-oxybenzotriazolyl (—OC₆H₄N₃); | | |
| # or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, | | |
| # where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl). | | |

**Acyl azides can also rearrange to isocyanates

In some embodiments, the reactive group further comprises a linker, L, in addition to the reactive functional moiety. The linker is used to covalently attach a reactive functional moiety to the BRS compound of the invention. When present, the linker is a single covalent bond or a series of stable bonds. Thus, the reactive functional moiety may be directly attached (where the linker is a single bond) to the BRS compound or attached through a series of stable bonds. When the linker is a series of stable covalent bonds the linker typically incorporates 1-20 nonhydrogen atoms selected from the group consisting of C, N, O, S, and P. In addition, the covalent linkage can incorporate a platinum atom, such as described in U.S. Pat. No. 5,714,327. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In an exemplary embodiment, the linker incorporates less than 15 nonhydrogen atoms and are composed of a combination of ether, thioether, thiourea, amine, ester, carboxamide, sulfonamide, hydrazide bonds and aromatic or heteroaromatic bonds. Typically the linker is a single covalent bond or a combination of single carbon-carbon bonds and carboxamide, sulfonamide or thioether bonds. The following moieties can be found in the linker: ether, thioether, carboxamide, thiourea, sulfonamide, urea, urethane, hydrazine, alkyl, aryl, heteroaryl, alkoxy, cycloalkyl and amine moieties. Examples of L include substituted or unsubstituted polymethylene, arylene, alkylarylene, arylenealkyl, or arylthio.

Any combination of linkers may be used to attach the reactive groups and the present compounds together, typically a compound of the present invention when attached to more than one reactive group will have one or two linkers attached that may be the same or different. The linker may also be substituted to alter the physical properties of the present compounds, such as solubility and spectral properties of the compound.

Where the reactive group is a maleimide or haloacetamide the resulting compound is particularly useful for conjugation to thiol-containing substances. Where the reactive group is a hydrazide, the resulting compound is particularly useful for conjugation to periodate-oxidized carbohydrates and glycoproteins. Where the reactive group is a silyl halide, the resulting compound is particularly useful for conjugation to silica surfaces, particularly where the silica surface is incorporated into a fiber optic probe subsequently used for remote ion detection or quantitation.

Solid Support

In an exemplary embodiment, the compounds of the invention are covalently bonded to a solid support. The solid support may be attached to the compound either through the BRS, or through a reactive group, if present. Even if a reactive group is present, the solid support may be attached through the BRS.

A solid support suitable for use in the present invention is typically substantially insoluble in liquid phases. Solid supports of the current invention are not limited to a specific type of support. Rather, a large number of supports are available and are known to one of ordinary skill in the art. Thus, useful solid supports include semi-solids, such as aerogels and hydrogels, resins, beads, biochips (including thin film coated biochips), multi-well plates (also referred to as microtitre plates), membranes, conducting and nonconducting metals and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the compounds of the invention. Useful reactive groups are disclosed below and are equally applicable to the solid support reactive functional groups herein.

A suitable solid phase support can be selected on the basis of desired end use and suitability for various synthetic protocols. For example, where amide bond formation is desirable to attach the compounds of the invention to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories).

In order to conjugate BRS/reactive group compounds to a solid support, the BRS/reactive group compounds of the invention are typically first dissolved in water or a water-miscible such as a lower alcohol, dimethylformamide (DMF), dimethylsulfoxide (DMSO), acetone, acetonitrile, tetrahydrofuran (THF), dioxane or acetonitrile. These preparations are well documented in Haugland, Molecular Probes, Inc. Handbook of Fluorescent Probes and Research Chemicals, (9th ed., September 2002) and Brinkley, Bioconjugate Chem., 3: 2 (1992). Conjugates typically result from mixing appropriate reactive compounds and the component to be conjugated in a suitable solvent in which both are soluble, using methods well known in the art, followed by separation of the conjugate from any unreacted component and by-products. These present compounds are typically combined with the component under conditions of concentration, stoichiometry, pH, temperature and other factors that affect chemical reactions that are determined by both the reactive groups on the compound and the expected site of modification on the component to be modified. These factors are generally well known in the art of forming bioconjugates (Haugland et al., “Coupling of Antibodies with Biotin”, The Protein Protocols Handbook, J. M. Walker, ed., Humana Press, (1996); Haugland “Coupling of Monoclonal Antibodies with Fluorophores”, Methods in Molecular Biology, Vol. 45: Monoclonal Antibody Protocols, W. C. Davis, ed. (1995)). For those reactive compounds that are photoactivated, conjugation requires illumination of the reaction mixture to activate the reactive compound. The labeled component is used in solution or lyophilized and stored for later use.

Synthesis

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention; it is not intended to define the scope of reactions or reaction sequences that are useful in preparing the compounds of the present invention.

BRS Synthesis (Avidin (BRC): Alexa Fluor 488 (Donor): HABA (Acceptor))

Alexa Fluor 488 dye-labeled avidin is obtained from Invitrogen Corp. (Catalog number A21370). HABA (2-(4-hydroxyphenylazo)benzoic acid) is obtained from Sigma Chemical Company (Catalog number H5126). A suitable stock solution of labeled avidin is prepared (e.g. 1 mg/mL) in PBS (50 mM phosphate, 150 mM NaCl, pH 7.4). An aliquot of this solution is diluted to 200 nM in PBS. HABA is added to a final concentration of 250 μM. BRS formation is nearly instantaneous upon addition of HABA. The complex is now ready for use. If desired, 0-10 μM additional unlabeled avidin (Invitrogen Corp.; catalog number A887) may be added. This will have the effect of shifting the analytical range of the assay towards higher biotin concentrations.

Methods of Use

The present invention also provides methods of using the compounds described herein for a wide variety of chemical, biological and biochemical applications.

Thus, in one aspect, the present invention provides a method for detecting biotin in a sample. The present method comprises:
i) contacting the carrier molecule with a BRS, to form a carrier molecule-biotin-BRS complex, wherein the BRS comprises:
   a) a biotin recognition compound (BRC) comprising a biotin-binding moiety;
   b) a donor moiety, wherein the donor moiety is covalently attached to the BRC at any portion other than within the biotin-binding moiety; and
   c) a biotin mimic compound that comprises an acceptor moiety, wherein the biotin mimic compound is non-covalently attached to the BRC at the biotin-binding moiety;
ii) illuminating the carrier molecule-biotin-BRS complex with an appropriate wavelength to form an illuminated carrier molecule-biotin-BRS complex; and,
iii) observing the illuminated carrier molecule-biotin-BRS complex, whereby the biotin on a carrier molecule is detected.

The method includes contacting the sample or carrier molecule with a BRS of the invention, which comprises a BRC, a donor moiety, and a biotin mimic moiety. Upon contact, biotin binds to the BRC at the biotin-binding moiety and dislodges the biotin mimic moiety. With the biotin mimic and acceptor moiety removed, the biotin-bound BRC forms a fluorescent product. In an exemplary embodiment, the method further comprises illuminating the fluorescent biotin-bound BRC product with an appropriate wavelength and observing the illuminated carrier molecule-biotin-BRS complex, whereby the biotin on a carrier molecule is detected.

Further embodiments include quantifying the number of biotins present on the carrier molecule. This method comprises the following steps:
i) releasing biotin from the carrier molecule;
ii) contacting the biotin with a BRS, whereby said biotin displaces an acceptor moiety on the BRS, thereby forming a biotin-BRS complex, wherein the BRS comprises:
   a) a biotin recognition compound (BRC) comprising a biotin-binding moiety;
   b) a donor moiety, wherein the donor moiety is covalently attached to the BRC at any portion other than within the biotin-binding moiety; and
   c) a biotin mimic compound that comprises an acceptor moiety, wherein the biotin mimic compound is non-covalently attached to the BRC at the biotin-binding moiety;
iii) illuminating the biotin-BRS complex with an appropriate wavelength to form an illuminated sample; and,
iv) observing the illuminated sample whereby the number of biotin molecules is determined.

In an exemplary embodiment, the BRC is avidin, streptavidin, captavidin, neutravidin, anti-biotin antibody or fragment thereof. In another embodiment the biotin mimic is (2-(4′-hydroxyazobenzene)benzoic acid) (HABA), Strep-Tag peptides or desthiobiotin. In one aspect the acceptor moiety is HABA in another aspect the acceptor moiety is covalently bonded to the biotin mimic moiety. In another aspect the biotin mimic is desthiobiotin labeled with and acceptor moiety such as Alexa Fluor 555.

In an exemplary embodiment the donor moiety is a xanthene, cyanine, borapolyazaindacene (BODIPY), coumarin, oxazine, acridinone, or styryl dyes. In one aspect the donor moiety is Alexa Fluor 488 dye or fluorescein. In a further aspect, the donor dye is encapsulated in a microparticle such as those disclosed in U.S. Pat. Nos. 5,326,692; 5,723,218; 5,573,909; and 6,005,113.

In a particular useful embodiment the BRS comprises avidin as the BRC, HABA as the biotin mimic and acceptor moiety and Alexa Fluor 488 dye as the donor moiety. In another equally useful embodiment the BRS comprises Streptavidin as the BRC, desthiobiotin as the biotin mimic, which is covalently conjugated to the acceptor moiety of Alexa Fluor 555 dye and the donor moiety is Alexa Fluor 488 dye.

The carrier molecule bonded to the biotin can be any of the carrier molecules known in the art. Exemplary carrier molecules include antigens, steroids, vitamins, drugs, haptens, metabolites, toxins, environmental pollutants, amino acids, peptides, proteins, nucleic acids, nucleic acid polymers, carbohydrates, lipids, and polymers. In particular, typical carrier molecules include peptide, protein, DNA, or RNA. Thus, in a further embodiment, the releasing step further comprises contacting the carrier molecule with a protease or nuclease. This step of utilizing an enzyme can be useful when the BRC is multivalent, such as avidin.

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide. In a preferred embodiment the carrier molecule is amino acid, a peptide, a protein, a polysaccharide, a nucleoside, a nucleotide, an oligonucleotide, a nucleic acid, a hapten, a psoralen, a drug, a hormone, a lipid, a lipid assembly, a tyramine, a synthetic polymer, a polymeric microparticle, a biological cell, cellular components, an ion chelating moiety, an enzymatic substrate or a virus. In another preferred embodiment, the carrier molecule is an antibody or fragment thereof, an antigen, an avidin or streptavidin, a biotin, a dextran, an antibody binding protein, a fluorescent protein, agarose, and a non-biological microparticle. Typically, the carrier molecule is an antibody, an antibody fragment, antibody-binding proteins, avidin, streptavidin, a toxin, a lectin, or a growth factor. Preferred haptens include biotin, digoxigenin and fluorophores.

Antibody binging proteins include, but are not limited to, protein A, protein G, soluble Fc receptor, protein L, lectins, anti-IgG, anti-IgA, anti-IgM, anti-IgD, anti-IgE or a fragment thereof.

In an exemplary embodiment, the carrier molecule is an enzymatic substrate that is an amino acid, peptide, sugar, alcohol, alkanoic acid, 4-guanidinobenzoic acid, nucleic acid, lipid, sulfate, phosphate, —CH₂OCOalkyl and combinations thereof. Thus, the enzyme substrates can be cleave by enzymes selected from the group consisting of peptidase, phosphatase, glycosidase, dealkylase, esterase, guanidinobenzotase, sulfatase, lipase, peroxidase, histone deacetylase, endoglycoceramidase, exonuclease, reductase and endonuclease.

In another exemplary embodiment, the carrier molecule is an amino acid (including those that are protected or are substituted by phosphates, carbohydrates, or C₁ to C₂₂ carboxylic acids), or a polymer of amino acids such as a peptide or protein. In a related embodiment, the carrier molecule contains at least five amino acids, more preferably 5 to 36 amino acids. Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Preferred protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. Typically, the protein carrier molecule is an antibody, an antibody fragment, avidin, streptavidin, a toxin, a lectin, or a growth factor. Exemplary haptens include biotin, digoxigenin and fluorophores.

In another exemplary embodiment, the carrier molecule comprises a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955) or other linkage. In another exemplary embodiment, the nucleotide carrier molecule is a nucleoside or a deoxynucleoside or a dideoxynucleoside.

Exemplary nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides.

In another exemplary embodiment, the carrier molecule comprises a carbohydrate or polyol that is typically a polysaccharide, such as dextran, FICOLL, heparin, glycogen, amylopectin, mannan, inulin, starch, agarose and cellulose, or is a polymer such as a poly(ethylene glycol). In a related embodiment, the polysaccharide carrier molecule includes dextran, agarose or FICOLL.

In another exemplary embodiment, the carrier molecule comprises a lipid (typically having 6-25 carbons), including glycolipids, phospholipids, and sphingolipids. Alternatively, the carrier molecule comprises a lipid vesicle, such as a liposome, or is a lipoprotein (see below). Some lipophilic substituents are useful for facilitating transport of the conjugated dye into cells or cellular organelles.

Alternatively, the carrier molecule is cells, cellular systems, cellular fragments, or subcellular particles. Examples of this type of conjugated material include virus particles, bacterial particles, virus components, biological cells (such as animal cells, plant cells, bacteria, or yeast), or cellular components. Examples of cellular components that can be labeled, or whose constituent molecules can be labeled, include but are not limited to lysosomes, endosomes, cytoplasm, nuclei, histones, mitochondria, Golgi apparatus, endoplasmic reticulum and vacuoles.

In an exemplary embodiment, the carrier molecule comprises a specific binding pair member wherein the present compounds are conjugated to a specific binding pair member and are used to detect a heavy metal ion in close proximity to the complimentary member of the specific binding pair. Exemplary binding pairs are set forth in Table 2.

TABLE 2
Representative Specific Binding Pairs

| | |
|---|---|
| antigen | antibody |
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DMA (RNA) | cDNA (cRNA)† |
| hormone | hormone receptor |
| ion | chelator |
| antibody | antibody-binding proteins |

*IqG is an immunoglobulin
^{†}cDNA and cRNA are the complementary strands used for hybridization

Compounds of the invention containing a reactive group and/or attached to a solid support are discussed in detail above and are equally applicable to the methods discussed herein.

In another exemplary embodiment, the present invention provides a method of detecting biotin in a sample or on a carrier molecule by using an immobilized BRS. The method includes combining the sample with a BRS of the invention covalently bonded to a solid support. The biotin in the sample is then allowed to react and dislodge the moiety (wither donor or acceptor, depending on the assay), which is noncovalently attached to the biotin-binding moiety, thus producing a detectable response and a biotin-BRS complex. In another exemplary embodiment, the method further comprises illuminating the fluorescent product with an appropriate wavelength so that the presence of biotin in the sample or on a carrier molecule is determined and its concentration in the sample is optionally quantified.

In methods of detecting biotin by using an immobilized BRS, the methods may further include, after forming the fluorescent product, rinsing the solid support to remove components of the sample other than the immobilized fluorescent product. In another exemplary embodiment, the methods may further provide, after forming the immobilized fluorescent product, detecting the immobilized fluorescent product. In a related embodiment, the immobilized fluorescent product is detected after rinsing the solid support.

Solid supports covalently bonded to a compound of the invention are discussed in detail above and are equally applicable to the methods discussed herein.

Rinsing the solid support typically functions to remove residual, excess or unbound materials from the solid support other than the immobilized fluorescent product. Any appropriate solution or series or solutions may be used to rinse the solid support. Exemplary solvents useful in the present invention include both polar and non-polar solvents. Thus, any appropriate organic solvent or aqueous solution is useful in the methods of the current invention.

Solutions of the compounds of the invention are prepared according to methods generally known in the art. The compounds of the invention are generally soluble in water and aqueous solutions having a pH less than or equal to about 6. Stock solutions of pure compounds of the invention, however, may be dissolved in organic solvent before diluting into aqueous solution or buffer. Exemplary organic solvents are aprotic polar solvents such as DMSO, DMF, N-methylpyrrolidone, acetone, acetonitrile, dioxane, tetrahydrofuran and other nonhydroxylic, completely water-miscible solvents. In general, the amount of BRS in solution is the minimum amount required to yield a detectable biotin presence signal within a reasonable time, with minimal background signal. The exact concentration of compound of the invention to be used is dependent upon the experimental conditions and the desired results, and optimization of experimental conditions is typically required to determine the best concentration to be used in a given application. The concentration typically ranges from nanomolar to micromolar. The required concentration is determined by systematic variation in compound concentration until satisfactory results are accomplished. The starting ranges are readily determined from methods known in the art for use of similar compounds under comparable conditions for the desired response.

Any suitable method of detection is useful in detecting fluorogenic or fluorescent compounds of the invention. In an exemplary embodiment, detection is achieved by illuminating the fluorogenic or fluorescent compounds at a wavelength selected to elicit a detectable optical response.

A detectable optical response means a change in, or occurrence of, a parameter in a test system that is capable of being perceived, either by direct observation or instrumentally. Typically the detectable response is a change in fluorescence, such as a change in the intensity, excitation or emission wavelength distribution of fluorescence, fluorescence lifetime, fluorescence polarization, or a combination thereof. The detectable optical response may occur throughout the sample or in a localized portion of the sample. The presence or absence of the optical response after the elapsed time is indicative of one or more characteristic of the sample. Comparison of the amount of the compound of the invention with a standard or expected response can be used to determine whether and to what degree a sample or carrier molecule possesses biotin.

Illumination

The BRSs of the invention are illuminated with a wavelength of light selected to give a detectable optical response, and observed with a means for detecting the optical response. Equipment that is useful for illuminating the present compounds and compositions of the invention includes, but is not limited to, hand-held ultraviolet lamps, mercury arc lamps, xenon lamps, lasers and laser diodes. These illumination sources are optically integrated into laser scanners, fluorescence microplate readers or standard or microfluorometers.

The compounds of the invention may, at any time after or during an assay, be illuminated with a wavelength of light that results in a detectable optical response, and observed with a means for detecting the optical response. Upon illumination, such as by an ultraviolet or visible wavelength emission lamp, an arc lamp, a laser, or even sunlight or ordinary room light, the fluorescent compounds, including those bound to the complementary specific binding pair member, display intense visible absorption as well as fluorescence emission. Selected equipment that is useful for illuminating the fluorescent compounds of the invention includes, but is not limited to, hand-held ultraviolet lamps, mercury arc lamps, xenon lamps, argon lasers, laser diodes, and YAG lasers. These illumination sources are optionally integrated into laser scanners, fluorescence microplate readers, standard or mini fluorometers, or chromatographic detectors. This fluorescence emission is optionally detected by visual inspection, or by use of any of the following devices: CCD cameras, video cameras, photographic film, laser scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, a fluorescence microscope or a fluorometer, the instrument is optionally used to distinguish and discriminate between the fluorescent compounds of the invention and a second fluorophore with detectably different optical properties, typically by distinguishing the fluorescence response of the fluorescent compounds of the invention from that of the second fluorophore. Where a sample is examined using a flow cytometer, examination of the sample optionally includes isolation of particles within the sample based on the fluorescence response by using a sorting device.

Sample Preparation

The end user will determine the choice of the sample and the way in which the sample is prepared. The sample includes, without limitation, any biological derived material that is thought to contain biotin. Alternatively, samples also include material in which a biotin (either isolated or as part of a carrier molecule) has been added.

The sample can be a biological fluid such as whole blood, plasma, serum, nasal secretions, sputum, saliva, urine, sweat, transdermal exudates, cerebrospinal fluid, or the like. Biological fluids also include tissue and cell culture medium wherein an analyte of interest has been secreted into the medium. Alternatively, the sample may be whole organs, tissue or cells from the animal. Examples of sources of such samples include muscle, eye, skin, gonads, lymph nodes, heart, brain, lung, liver, kidney, spleen, thymus, pancreas, solid tumors, macrophages, mammary glands, mesothelium, and the like. Cells include without limitation prokaryotic cells and eukaryotic cells that include primary cultures and immortalized cell lines. Eukaryotic cells include without limitation ovary cells, epithelial cells, circulating immune cells, β cells, hepatocytes, and neurons.

In many instances, it may be advantageous to add a small amount of a non-ionic detergent to the sample. Generally the detergent will be present in from about 0.01 to 0.1 vol. %.

Illustrative non-ionic detergents include the polyoxyalkylene diols, e.g. Pluronics, Tweens, Triton X-100, etc.

Kits

The invention provides a kit for detecting biotin on a carrier molecule or in a sample, wherein said kit comprises:
i ) a BRS; wherein the BRS comprises:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety; and
ii) instructions on the use of the BRS.

In an exemplary embodiment, the kit further comprises a reaction buffer, a carrier molecule, positive control. In another exemplary embodiment, the kit further comprises biotin or a biotin-carrier molecule conjugate as a positive control. In another exemplary embodiment, the carrier molecule is a member selected from peptide and a protein. In another exemplary embodiment, the kit is further comprises a protease. In another exemplary embodiment, the carrier molecule is a member selected from DNA and RNA. In another exemplary embodiment, kit further comprises a nuclease.

The materials and methods of the present invention are further illustrated by the examples which follow. These examples are offered to illustrate, but not to limit, the claimed invention.

A detailed description of the invention having been provided above, the following examples are given for the purpose of illustrating the invention and shall not be construed as being a limitation on the scope of the invention or claims.

### EXAMPLES

### Example 1

### Constructing Biotin Standard Curves

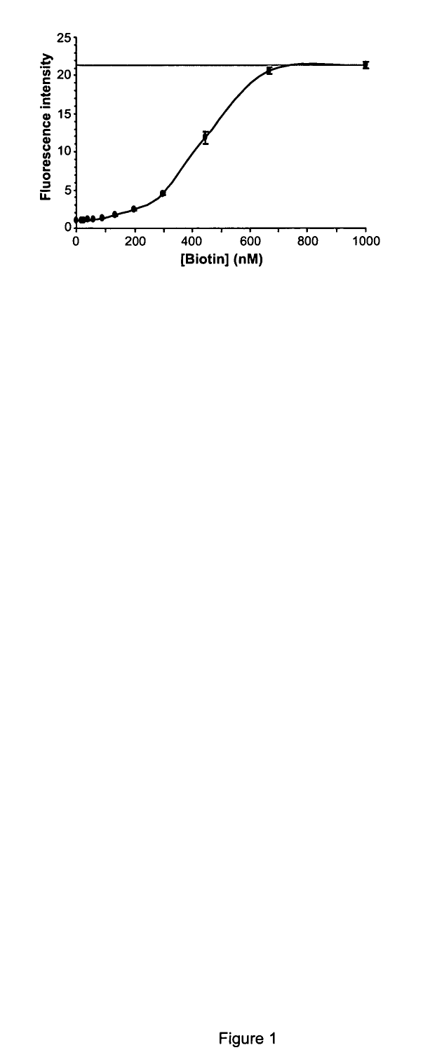

Biotin standard curves were obtained by titrating a solution containing 200 nM Alexa Fluor 488-labeled avidin and 250 μM HABA in PBS (50 mM phosphate, 150 mM NaCl, pH 7.4). Biotin was added from an aqueous stock solution to give final concentrations of 0-1000 nM in 100 μL samples. Fluorescence of samples was measured in a microplate reader (CytoFluor 4000, PerSeptive Biosystems, excitation/emission=485/530 nm). The results are presented in FIG. 1.

The data illustrates the ˜21-fold increase in fluorescence signal upon complete displacement of HABA. This is a larger optical signal than can be obtained using donor quenching by the protein matrix as the signal-generating mechanism (see Nargassi et al., Meth. Enzymol. 122:67-72 (1986)). The fluorescence signal increase upon biotin binding for most donor-labeled avidin conjugates is typically about 2-fold and rarely greater than 10-fold. The limit of detection is about 20 nM biotin, about 100-fold lower than the conventional spectrophotometric HABA assay (˜2 μM limit of detection).

### Example 2

### Biotin Standard Curves with Concentration Range Adjustments

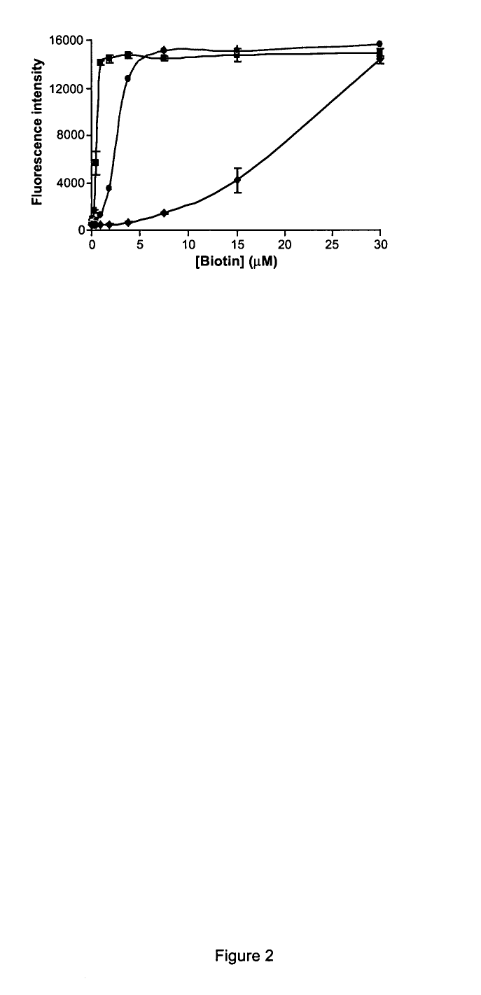

The analytical range of the assay was shifted to match the concentration range of input samples by adding unlabeled avidin to the sample. Several titrations were conducted according to the general conditions described in Example 1. The results are presented in FIG. 2. Three standard curves were recorded. The first curve (square) contained no added unlabeled avidin. The second curve (circle) had 0.8 μM unlabeled avidin. The third curve (diamond) had 7.0 μM unlabeled avidin.

### Example 3

### Measuring the Extent of Biotinylation on a Protein with and without Enzymatic Digestion

Assays that use avidin (or streptavidin, as well as some of their derivatives) as the BRC share a common limitation when used for analysis of multiple-biotinylated proteins and nucleic acids. Conformational restraints imposed by the macromolecular framework restricts the access of the biotin labels to the BRC binding sites, resulting in underestimation of the biotin concentration. The conventional solution to this problem for proteins, adopted here, is to pre-treat samples with protease prior to analysis.

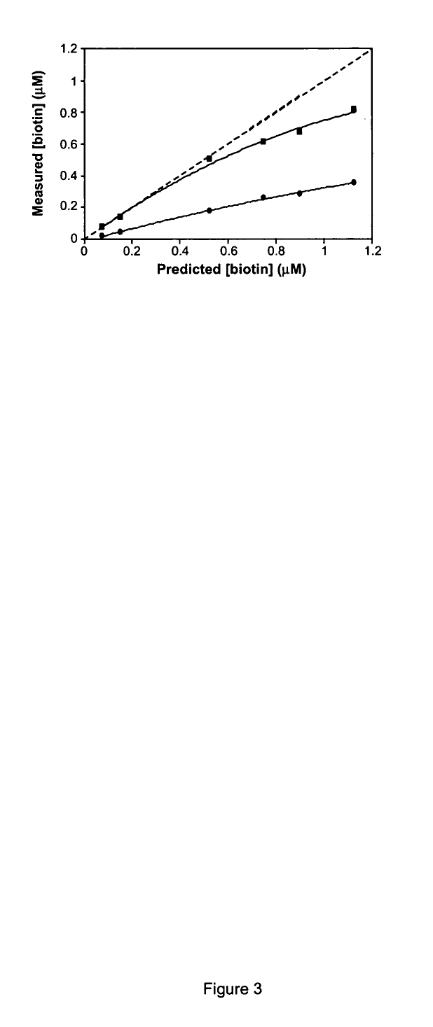

Samples containing 0.01-0.15 μM BRC (biotin-XX goat anti-mouse IgG, (Molecular Probes, Inc., B2763)) were subjected to enzymatic digestion via proteinase K (1 U/mL) digestion. Proteins were subject to digestion overnight. Fluorescence of samples was measured in a microplate reader (CytoFluor 4000, PerSeptive Biosystems, excitation/emission=485/530 nm). The fluorescence intensity data was collected and converted to corresponding biotin concentrations by reference to a standard curve (e.g. FIG. 1 or FIG. 2). These values are plotted relative to predicted biotin concentrations obtained from the input protein concentrations multiplied by 7.6 biotin:IgG determined by mass spectrometry (See Example 4). The results are presented in FIG. 3. The dotted line indicates the intersection of predicted and measured values. The square line represents a sample treated with proteinase K while the circle line represents a sample that was not treated with proteinase K. Because the square line more closely approximates the dotted line, enzymatic digestion pre-treatment provides a more accurate description of the extent of biotinylation on a carrier molecule.

### Example 4

### Reference Analysis of Protein Biotinylation by MALDI-TOF

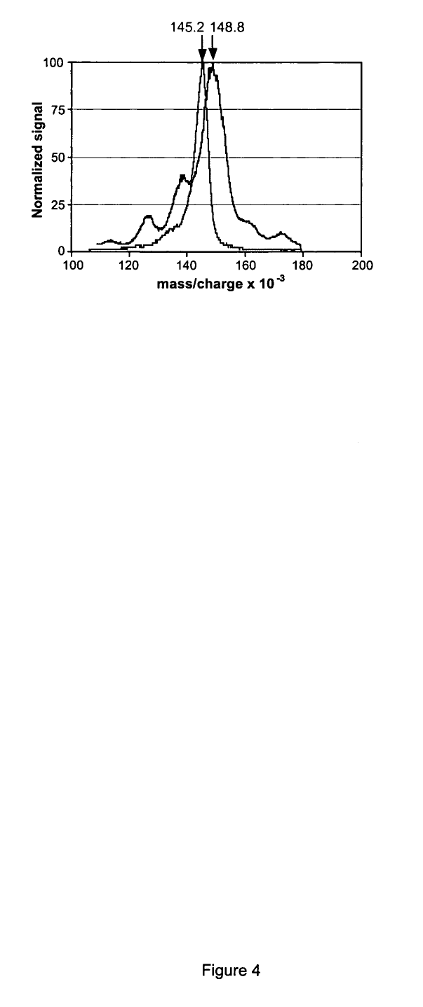

A 10 pmol sample of unlabeled goat anti-mouse IgG and a 10 pmol sample of biotin-XX goat anti-mouse IgG in water were diluted 1:1 into 10 mg/mL sinapinic acid in 70:30 (v/v) MeCN:H₂O. Samples (0.5 uL) of each solution were spotted and allowed to dry. The samples were analyzed in positive reflectron mode on a MALDI Axima CFR spectrometer (Kratos, Manchester, UK). The results are presented in FIG. 4. The peak separation of the spectrograms for the labeled proteins (“148.8”) and unlabeled proteins (“145.2”) corresponds to 3.6 KDa, equivalent to ˜7.6 biotin-XX labels (molecular weight=470) per IgG.

### Example 5

### Measuring the Extent of Biotinylation on a Nucleic Acid with and without Enzymatic Digestion

Assays that measure the extent of biotinylation share a common limitation when used for analysis of multiple-biotinylated proteins and nucleic acids. Conformational restraints imposed by the macromolecular framework restricts the access of the biotin labels to the BRC binding sites, resulting in underestimation of the biotin concentration. The conventional solution to this problem for nucleic acids, adopted here, is to pre-treat samples with nuclease prior to analysis.

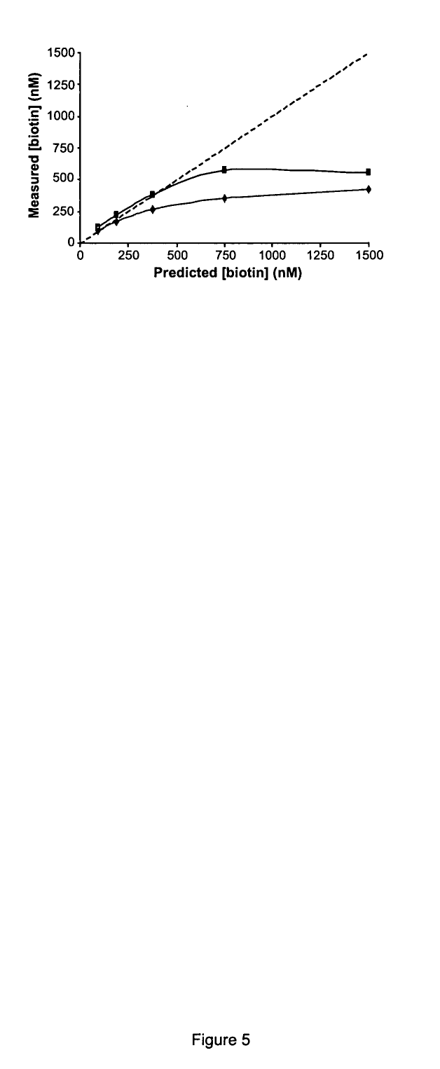

A triply-biotinylated oligonucleotide 11-mer (G([biotin]ATCAT[biotin]ACAGT[biotin]) was custom synthesized by Qiagen. Samples of the oligonucleotide were analyzed with or without overnight pre-treatment with micrococcal nuclease (˜100 U/sample) in 100 mM sodium borate pH 9.3, 2 mM CaCl₂. The fluorescence intensity data shown were converted to corresponding biotin concentrations by reference to a standard curve. These values are plotted relative to predicted biotin concentrations obtained from the input oligonucleotide concentrations multiplied by 3 biotin:oligonucleotide. The dotted line indicates the intersection of predicted and measured values. The square line represents a sample treated with micrococcal nuclease while the circle line represents a sample that was not treated with micrococcal nuclease. The results are presented in FIG. 5.

### Example 6

### Use of Alternative Acceptor Donors

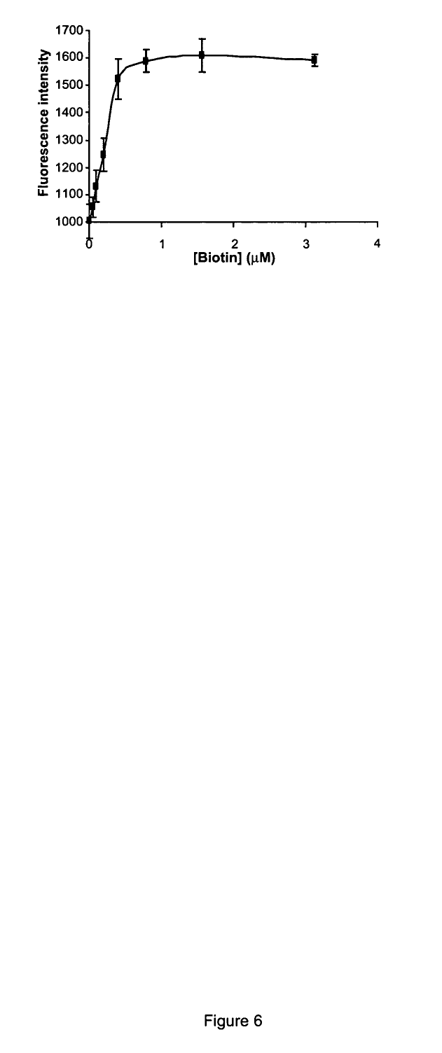

The general assay principle described in Example 1 can be extended beyond the specific BRC, donor, and acceptor grouping of avidin (BRC), Alexa Fluor 488 dye (donor), and HABA (acceptor). FIG. 6 shows a biotin standard curve obtained using a detection complex comprising 200 nM Alexa Fluor 488-labeled avidin and 1 μM Alexa Fluor 555-labeled desthiobiotin.

Donor-labeled avidin conjugates are required for use in conjunction with HABA as the binding affinity of HABA for streptavidin is about 20-fold lower. Donor-labeled conjugates of the biotin analog desthiobiotin with spectral properties permitting efficient FRET quenching of the donor can be used with equal effectiveness in conjunction with avidin and streptavidins. The binding of desthiobiotin to avidin (K_{d}˜0.1 μM) is significantly stronger than that of HABA (K_{d}˜6 μM) but weak enough to be readily displaced by biotin.

Donor-labeled conjugates of the biotin analog desthiobiotin with spectral properties permitting efficient FRET quenching of the donor can be used with equal effectiveness in conjunction with avidin and streptavidins. The binding of desthiobiotin to avidin (K_{d}˜0.1 μM) is significantly stronger than that of HABA (K_{d}˜6 μM) but weak enough to be readily displaced by biotin.

### Example 7

### Protocol for a Protein Biotinylation Detecting Kit

The sample kit contains 25 Units of Protease (Component A); 5×5 mL, lyophilized Fluorescent biotin detection reagent (Component B); 900 μL of 200 μM D-Biotin (Component C); 23 mg of avidin (Component D); and 10 mL of 10×PBS buffer (Component E). These components should be stored at −20° C. until required for use.

a) Stock Solution Preparation

Allow components to warm to room temperature before preparing the various stock solutions. First, prepare 20 mL of 1× working solution of Reaction Buffer by adding 2 mL of 10× reaction buffer stock solution to 18 mL of deionized water. This 1× reaction buffer should be sufficient for approximately 100 assays of 100 μL each with 10 mL excess for making stock solutions and dilutions. Next, prepare a 500 μM stock solution of avidin by dissolving the entire tube of component D in 700 μL of 1× reaction buffer. Store at 2-6° C. Next, prepare a 100 U/mL protease stock solution by adding 250 μL of 1× reaction buffer to the entire tube of Component A. Store the 200 μM D-biotin at 2-6° C. Then finally, as needed, resuspend the lyophilized Fluorescent biotin detection reagent (Component B) in 5 mL 1× reaction buffer. Store at 4° C. for a maximum of two weeks.

b) Protease Digestion of Biotinylated Protein

Dissolve the biotinylated protein sample in 1× reaction Buffer if not already in solution. Add 1/100^{th} of the sample volume of Component A to the biotinylated sample. For example, if the sample is 100 μL, add 1 μL of 100 U/ml protease (Component A). Incubate overnight at 25-38° C. This step is optional, but recommended in order to expose the biotin groups on the protein. By making the biotin fully accessible to the fluorescent biotin detection reagent through preasea degradation the accuracy of the assay is maximized.

c) Biotin Assay

The following protocol describes a biotin assay in a total volume of 100 μL per well. The volumes recommended here are sufficient for ˜100 assays. The kit provides sufficient material for ˜500 assays. First, prepare a biotin standard curve. Add 6.8 μL of 200 μM D-biotin (Component C) to 843 μL of 1× reaction buffer to produce a 1.6 μM biotin solution. Serially dilute the 1.6 μM biotin across a 96 well microplate in triplicate rows of 50 μL of 1× reaction buffer. Use 1× reaction buffer without biotin as a negative control in the final column. Please note that the biotin concentrations will be two fold lower in the final reaction volume. Then, dilute the protease-digested biotinylated samples in 1× reaction buffer to a concentration where the biotin concentration of the sample is believed to be in the dynamic range of 20-800 nM biotin. Please note that these concentrations will be two fold lower in the final reaction volume. If the biotinylation of the protein is unknown, a wide range of dilutions is recommended in order that a few may land in the dynamic range of the assay. NOTE: If desired, the dynamic range can be shifted upwards (less sensitive) by ‘cutting’ the Fluorescent biotin detection reagent (Component B) with avidin (Component D). For example cutting the Fluorescent biotin detection reagent with 14.0 μM avidin shifts the dynamic range to ˜2-30 μM biotin. Pipette 50 μL of the diluted samples into separate empty wells of the microplate. Begin the reactions by adding 50 μL of the Fluorescent biotin detection reagent (Component B) to each microplate well containing the samples. Immediately measure the fluorescence in a microplate reader using typical fluorescein wavelengths (excitation of ˜485 nm and an emission of ˜530 nm). If the reactions are not read soon (<15 minutes) after addition of the Fluorescent biotin detection reagent (Component B), the protease will affect the assay. If for some reason a longer delay is required after addition of the Fluorescent biotin detection reagent, a protease inhibitor should be added.

d) Data Analysis

Plot the Biotin Standard Curve Fluorescence (x axis) over the biotin concentration (y-axis). Use the quadratic fit on Excel to find the quadratic equation of the Biotin Standard Curve from zero to the upper limit of the final biotin concentration. Insert the measured relative fluorescence units as your Y value and solve for X. This should equal the biotin concentration of the sample. An alternative method to using the quadratic fit on Excel is to plot the Biotin standard curve from zero to the upper limit of the final biotin concentration, and draw a straight line from the two points on the standard curve that surround the biotinylated macromolecule fluorescence. Use the equation of the line to find the biotin concentration on the macromolecule by inserting the measured fluorescence as the Y value and solving for X. Yet another alternative to using the quadratic fit on Excel is to create a Hill Plot of the biotin titration. Do this by setting the LOG of the biotin concentrations from 0.025 μM to 0.8 μM of the biotin titration as the X-axis. Set the Y-axis equal to LOG((F−Fₘᵢₙ)/(Fₘₐₓ−F)) using F as the fluorescence corresponding to the biotin concentration used in the standard curve. Plot these points on a graph. This should result in a fairly straight line. (If not we recommend one of the other methods.) Then using the measured fluorescence from the biotinylated sample as F, solve for Y. Use that Y value to find X with the equation of the line from the HILL Plot. This should equal the biotin concentration of the sample. Once the biotin concentration of the macromolecule has been calculated, divide that number by the concentration of the macromolecule used to get the degree-of-labeling.

### Example 8

### Protocol for a Nucleic Acid Biotinylation Detecting Kit

The sample kit contains 25 Units of Nuclease (Component A); 5×5 mL, lyophilized Fluorescent biotin detection reagent (Component B); 900 μL of 200 μM D-Biotin (Component C); 23 mg of avidin (Component D); and 10 mL of 10×PBS buffer (Component E); and 2× nucleic acid digestion buffer (Component F). These components should be stored at −20° C. until required for use. These components should be stored at −20° C. until required for use.

a) Stock Solution Preparation

Allow components to warm to room temperature before preparing the various stock solutions. Prepare 20 ml of 1× working solution of Reaction Buffer, by adding 2 mL of 10× Reaction Buffer Stock solution to 18 ml of deionized water. This 1× Reaction Buffer should be sufficient for approximately 100 assays of 100 μL each with 10 mL excess for making stock solutions and dilutions. Prepare a 500 μM stock solution of avidin by dissolving the entire tube of component D in 700 μL of 1× reaction buffer. Store at 2-6° C. Store the 200 uM D-Biotin (Component C) at 2-6° C. As needed, resuspend each vial of nuclease (Component A) in 50 μL 2× nucleic acid digestion buffer (Component F). This is a 100 × stock and sufficient for treating about 5 mL of diluted sample (see step 2.2). For short-term use, store on ice or 2-6° C. overnight. For longer storage aliquot and freeze at −20° C. until required for use. Do not subject to repeat freeze/thaw cycles

b) Nuclease Digestion of Multiply Biotinylated DNA/RNA

Dilute the biotinylated nucleic acid samples with an equal volume of 2× nucleic acid digestion buffer (Component F). For example, dilute a 50 μL sample with 50 μL of 2× nucleic acid digestion buffer. Add 1/100^{th} of the volume of resuspended nuclease (Component A) to the diluted samples. For example, to a 100 μL diluted sample, add 1 μL of resuspended nuclease. Let incubate for at least 30 minutes at 37° C.

c) Biotin Assay

The following protocol describes a biotin assay in a total volume of 100 μL per well. The volumes recommended here are sufficient for ˜100 assays. The kit provides sufficient material for ˜500 assays. Prepare a biotin standard curve: Add 6.8 μL of 200 μM D-biotin (Component C) to 843 μL of 1× reaction buffer to produce a 1.6 μM biotin solution. Serially dilute the 1.6 μM biotin across a 96 well microplate in triplicate rows of 50 μL of 1× reaction buffer. Use 1× reaction buffer without biotin as a negative control in the final column. Please note that the biotin concentrations will be two fold lower in the final reaction volume. Dilute the nuclease-digested biotinylated samples in 1× reaction buffer to a concentration where the biotin concentration of the sample is believed to be in the dynamic range of 20-800 nM biotin. Please note that these concentrations will be two fold lower in the final reaction volume. If the biotinylation of the nucleic acids is unknown, we recommend a wide range of dilutions in order that a few may land in the dynamic range of the assay. NOTE: If desired, the dynamic range can be shifted upwards (less sensitive) by ‘cutting’ the Fluorescent biotin detection reagent (Component B) with avidin (Component D). For example cutting the Fluorescent biotin detection reagent with 14.0 μM avidin shifts the dynamic range to ˜2-30 μM biotin. Pipette 50 μL of the diluted samples into separate empty wells of the microplate. We recommend each sample concentration be performed in triplicate. Begin the reactions by adding 50 μL of the Fluorescent biotin detection reagent (Component B) to each microplate well containing the samples. Immediately measure the fluorescence in a microplate reader using typical fluorescein wavelengths (excitation of ˜485 nm and an emission of ˜530 nm).

d) Data Analysis

Plot the Biotin Standard Curve Fluorescence (x axis) over the biotin concentration (y-axis). Use the quadratic fit on Excel to find the quadratic equation of the Biotin Standard Curve from zero to the upper limit of the final biotin concentration. Insert the measured relative fluorescence units as your Y value and solve for X. This should equal the biotin concentration of the sample. An alternative method to using the quadratic fit on Excel is to plot the Biotin standard curve from zero to the upper limit of the final biotin concentration, and draw a straight line from the two points on the standard curve that surround the biotinylated macromolecule fluorescence. Use the equation of the line to find the biotin concentration on the macromolecule by inserting the measured fluorescence as the Y value and solving for X. Yet another alternative to using the quadratic fit on Excel is to create a Hill Plot of the biotin titration. Do this by setting the LOG of the biotin concentrations from 0.025 μM to 0.8 μM of the biotin titration as the X-axis. Set the Y-axis equal to LOG((F−Fₘᵢₙ)/(Fₘₐₓ−F)) using F as the fluorescence corresponding to the biotin concentration used in the standard curve. Plot these points on a graph. This should result in a fairly straight line. (If not we recommend one of the other methods.) Then using the measured fluorescence from the biotinylated sample as F, solve for Y. Use that Y value to find X with the equation of the line from the HILL Plot. This should equal the biotin concentration of the sample. Once you have calculated the biotin concentration of the macromolecule, divide that number by the concentration of the macromolecule used to get the degree-of-labeling.

The preceding examples can be repeated with similar success by substituting the specifically described BRS compounds of the preceding examples with those generically and specifically described in the forgoing description. One skilled in the art can easily ascertain the essential characteristics of the present invention, and without departing from the spirit and scope thereof, can make various changes and modifications of the invention to adapt to various usages and conditions. All publications, patents, and patent applications cited herein are hereby incorporated by reference in their entirety for all purposes.

1. A biotin recognition sensor (BRS) comprising:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety.

2. The compound according to claim 1, wherein the BRC is avidin, streptavidin, captavidin, neutravidin, anti-biotin antibody or fragment thereof.

3. The compound according to claim 1, wherein the biotin mimic is (2-(4′-hydroxyazobenzene)benzoic acid) (HABA), Strep-Tag peptides or desthiobiotin.

4. The compound according to claim 1, wherein the acceptor moiety comprises substituted (2-(4′-hydroxyazobenzene)benzoic acid) (HABA), unsubstituted (2-(4′-hydroxyazobenzene)benzoic acid) (HABA), a quencher moiety or a dye moiety.

5. The compound according to claim 1, wherein the biotin mimic comprises a dye labeled desthiobiotin or a quencher labeled desthiobiotin.

6. The compound according to claim 5, wherein the dye labeled desthiobiotin is Alexa Fluor 555 dye labeled desthiobiotin.

7. The compound according to claim 1, wherein the donor moiety is a xanthene, cyanine, borapolyazaindacene (BODIPY), coumarin, oxazine, acridinone, or styryl dyes.

8. The compound according to claim 1, wherein the donor moiety is Alexa Fluor 488 dye or fluorescein.

9. The compound according to claim 1, wherein the donor moiety is encapsulated within a microparticle.

10. The compound according to claim 1, wherein the BRC is avidin, the acceptor moiety is (2-(4′-hydroxyazobenzene)benzoic acid) (HABA) and the donor moiety is Alexa Fluor 488 dye.

11. The compound according to claim 1, wherein the BRC is streptavidin, the acceptor moiety is Alexa Fluor 555 dye labeled desthiobiotin and the donor moiety is Alexa Fluor 488 dye.

12. A biotin recognition sensor (BRS) comprising:
a) a biotin recognition compound (BRC) comprising a biotin-binding moiety;
b) an acceptor moiety, wherein the acceptor moiety is covalently attached to the BRC at any portion other than within the biotin-binding moiety; and
c) a biotin mimic compound that comprises a donor moiety, wherein the biotin mimic compound is non-covalently attached to the BRC at the biotin-binding moiety.

13. A method of detecting biotin on a carrier molecule, the method comprising:
i) contacting the carrier molecule with a BRS, to form a carrier molecule-biotin-BRS complex, wherein the BRS comprises:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety;
ii) illuminating the carrier molecule-biotin-BRS complex with an appropriate wavelength to form an illuminated carrier molecule-biotin-BRS complex; and, iv) observing the illuminated carrier molecule-biotin-BRS complex, whereby the biotin on a carrier molecule is detected.

14. The method according to claim 13, wherein the observing comprises quantifying the number of biotins present on the carrier molecule.

15. The method according to claim 13, wherein the carrier molecule is a peptide, protein, DNA, or RNA.

16. A method for determining the number of biotin molecules on a carrier molecule, the method comprising:
i) releasing biotin from the carrier molecule;
ii) contacting the biotin with a BRS, whereby said biotin displaces an acceptor moiety on the BRS, thereby forming a biotin-BRS complex, wherein the BRS comprises:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety;
iii) illuminating the biotin-BRS complex with an appropriate wavelength to form an illuminated sample; and,
v) observing the illuminated sample whereby the number of biotin molecules is determined.

17. The method according to claim 16, wherein the carrier molecule is a peptide, a protein, DNA or RNA.

18. The method according to claim 16, wherein the releasing step further comprises contacting the carrier molecule with a protease or nuclease.

19. A kit for detecting biotin on a carrier molecule, wherein said kit comprises:
i) a BRS; wherein the BRS comprises:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety; and
ii) instructions on the use of the BRS.

20. A composition for the quantitative determination of biotin in a sample, wherein said composition comprises:
i) a sample;
ii) a BRS; and wherein the BRS reacts with the biotin to form an amount of a fluorescent product proportional to the amount of biotin in the sample, and wherein the BRS comprises:
a) a biotin recognition compound (BRC) comprising a biotin-binding moiety;
b) a donor moiety, wherein the donor moiety is covalently attached to the BRC at any portion other than within the biotin-binding moiety; and
c) a biotin mimic compound that comprises an acceptor moiety, wherein the biotin mimic compound is non-covalently attached to the BRC at the biotin-binding moiety.
